(19) United States
(12) Patent Application Publication
(10) Pub. No.: US 2026/0183088 A1
(43) Pub. Date: Jul. 2, 2026

(54) Extraoral Orthopedic Device for the Protraction of the Upper and Lower Human Jaws

(71) Applicants: Stylianos Koutzoglou, Rethymno (GR); Eleni Koutzoglou, Rethymno (GR); Despoina Koutzoglou, Rethymno (GR)

(72) Inventors: Stylianos Koutzoglou, Rethymno (GR); Eleni Koutzoglou, Rethymno (GR); Despoina Koutzoglou, Rethymno (GR)

(21) Appl. No.: 19/131,570

(22) PCT Filed: May 24, 2023
(86) PCT No.: PCT/GR2023/000022

§ 371 Date: Jul. 1, 2025

(30) Foreign Application Priority Data
Nov. 28, 2022 (GR) 20220100971

(51) Int. Cl. A61C 7/06; A61C 7/10

(52) U.S. Cl. CPC A61C 7/06; A61C 7/10

(57) ABSTRACT

An extraoral orthopedic device (1) for the direct protraction of the maxilla and the direct or indirect protraction of the mandible, comprising: two metallic girders, one mouth girder (4), which is coupled through traction means (5) with an intraoral mechanism (6) and one connecting girder (3), which couples the said mouth girder (4) with a skeletal support wreath (2), placed and supported on the neurocranium (9) by means of its precision to the anatomy of it and the two elastic fixation straps (7) passing around the shoulders and through the axillae (8), wherein a) said skeletal support wreath (2), individually designed using the 3D digital imprint of a person's head and a computer, comprises first means of adjustment and support (12, 13, 14, 15) in order to ensure the optimal placement of the said connecting girder (3) and first means of support (17) aiming at its fixation to the neurocranium (9) by means of the straps (7), b) the said mouth girder (4) comprises first means of adjustment (25) and support (21, 22, 24) aiming at its ergonomic placement into the said connecting girder (3), thus ensuring optimal direction of the traction means (5) individualized to each person.

This invention is an extraoral device for the direct protraction of the maxilla through forward, downward or upward adjustments, as each case requires, as well as for the direct or indirect protraction of the mandible, where it is necessary.

Through this extraoral device, direct traction is applied to the maxilla immediately after its “rapid expansion” [1-4] using the traditional or alternate way [5-7], with elastic bands which are attached to the extraoral device and some classic mechanisms of “rapid maxillary expansion”, as in: [1-4] or through skeletal support, as in: [8]. The mandible can also be protracted both directly or indirectly in patients with a skeletal Class II malposition of the jaws, when the extraoral device is combined with intraoral mechanisms in the maxilla and/or in the mandible.

A. Until now, the commonly used extraoral devices for the protraction of the maxilla, are the “facemasks” designed by Delaire [9, 10] and Pettit [11] which are related only to the skeletal Class III malocclusion. In both of these devices, the forehead and chin are used as support in order to apply elastic forces, which currently amount to approximately 400 gr per side. In this way, the protraction of the maxilla is achieved (action), while the simultaneous pressure (reaction) on the temporomandibular joint tissues (particularly on the condyle, articular disc and fossa) could lead to a TMJ derangement in patients predisposed to that. Extraoral orthopedic devices used in the same way (support on the frontal bone and the chin) are the Turley “mask” [12] and the Face Mask/Reverse-pull Headgear Tübinger Model [13]. The “Sky Hook” headgear [14], which is supported on the cranial bones, parietal and occipital, as well as on the chin, is also used for the protraction of the maxilla after its “rapid expansion”. The elastic forces applied to the maxilla originate mostly from its chin support.

Historically seen, in the middle of the last century, there was an attempt to treat the maxillary deficiency using a helmet combined with a chin strap [15] [U.S. Pat. No. 2,325,300 A]. This invention, however, displays the same negative outcomes as mentioned above, in our opinion.

B. The Grummons facemask [16] uses the forehead as support and, instead of the chin, the infraorbital zygomatic area. This type of support differentiates this device from all of the above-mentioned. The face mask produced by the Leone company [17] uses the same support-area. While these devices are used with the aim to protract the maxilla after its rapid expansion, the therapy results are highly controversial because of the use of the infraorbital zygomatic support areas. These areas of the face comprise the zygomaticomaxillary sutures, which are subjected to a reactive pressure, thus generating the maxillary pulling action.

C. From time to time, devices have appeared for the maxillary protraction which use as skeletal anchorage only the frontal bone of the skull. The “maxillary modified protraction headgear” [18] is one of them. The only advantage of this device is its support. The non-use of the mandible, as support for the protraction of the maxilla, eliminates the possible side-effect of TMJ derangement. The industrial production, as well as the practical use of this device, are enormously impeded by the personalized bending of the extraoral and intraoral wires separately in each patient, aiming, firstly, at an easy insertion of the intraoral wires to the tubes of a concrete intraoral appliance and secondly, at avoiding the well-known side-effects during maxillary protraction.

D. Nowadays, intraoral orthopedic devices of skeletal support are widely used in the treatment of Angle Class III malocclusion. Mini-plates [19] or titanium mini-plates [8] are surgically attached to the infra-zygomatic region in the maxilla and between the permanent canines and lateral incisors in the mandible [19] or underneath the permanent lower incisor apices [8]. The skeletal anchorage in the maxilla could also comprise apart from teeth two palatal mini-screws [8], which are incorporated into an appliance (Hybrid-hyrax RME) used for “rapid maxillary expansion” and protraction of the maxilla, or could be titanium mini-plates placed on the infra-zygomatic crest above the buccal roots of the first permanent molar [19]. Growth repression of the mandible occurs because of the simultaneous backward compression of the chin from these devices. The glenoid fossa is transformed with a corresponding displacement of the condyle [20]. These anatomic changes are not required in all Angle Class III malocclusion cases, such as in patients who present a physiological growth in their mandible in conjunction with maxillary retrognathism due to the size or position of the maxilla [21]. This is commonly the case in cleft lip and palate patients. In the cases where these anatomic changes could be a positive outcome, their medical usefulness is lost because of the compression of the TMJ tissues, which could lead to a TMJ dysfunction. Extraoral orthopedic devices supported on the frontal bone and the chin, in combination with titanium infra-zygomatic mini-plates, have been used for the maxillary protraction [22]. In all these three types of devices in category D, the mandible is pushed backwards, while the maxilla is protracted. In addition to the risk of creating a TMJ derangement, as already mentioned above (category A devices), there are a lot of cases in which the mandible is positioned properly in the face. Nevertheless, all these types of devices operate in a compensatory manner, pushing the mandible backwards, modifying its growth and influencing its function and the aesthetic of the face adversely.

Functionally, besides the side-effects that can develop from the compression of the TMJs, it should be pointed out that the backward push of the mandible, followed by the tongue, could contribute to future problems with sleep apnea. Not forgetting, that all orthodontic appliances, which are used in the therapy of sleep apnea, are designed to bring the mandible mainly forward, with the tongue following this displacement of the mandible with simultaneous relief of the airway on the oropharynx level.

E. The “orthodontic appliance” [23] [U.S. Pat. No.: 5,158,451], the “Mandibular advancement” [24] [U.S. PATENT 2012/0040301A1], the “orthopedic device for the protraction of the maxillary arc” [25] [WO2017089971 (A1)], the “maxillary protraction device” [26] [US2018028282 (A1)], the “extraoral orthopedic device for the direct protraction of the maxilla and the indirect protraction of the mandible” [27] [WO2021090035A1] and [28] [US 2004/0199094A1] are specifically designed so that in use they avoid TMJ compression and increase the volume of the oral cavity.

The device described in FIGS. 1-16, provides excellent stability, greater efficiency, ergonomics and flexibility in its application and use, due to:

1. The true anatomical design of the fully individualized skeletal support wreath on the neurocranium and the perfect stabilization of the wreath on it because of its excellent fit and through the two elastic fixation straps (7) of the same length, passing under the axillae and around the shoulders reconnecting with their cranial ends, both of which pull and immobilize the wreath downwards, in order to avoid its dislodgment when the device (1) is in use. Generally, three methods exist through which the true cranial shape can be gained: CT (invasive method), impression of the cranium using a proper impressive material and scanning using a 3D-scanner, as described below (non-invasive method). The method employed in the herein device, 3D-scanning, is very accurate and the least invasive. In the device of invention [28], customization is referred to, but with no mention of any method. Secondly, the issue of dislodgment in the design of this device [28] is apparent, because of the lack of any element on the rear side of the device in order to provide the requisite opposite moment. Overall, there is a distinct lack of clarity. Furthermore, the invention [28] mentions “inflatable bladders” and “internally disposed shaping members and compressible liner” as a general idea, in order to achieve comfort and customization. Additionally, “the closure means”, which is presented as a form of customization could produce some pain in the pressure temporal area. Regarding all of these points, there is a crucial lack of detail and clarity. Firstly, customization cannot be achieved without using one of the three above-mentioned methods and secondly, in the herein presented invention there is, apart from scanning, the use of an adjustable bag containing air, or air and a soft material, or liquid, or air and liquid. Air or liquid intake or outtake is performed through the valve (32) to such an amount in order to create a comfortable cushion covering the skull relief individually.

2. The construction material of the connecting and the mouth girders is an aluminum alloy. Using this material, the girders are both very light and resistant to corrosion, especially through their anodizing. The aluminum alloy is a nickel-free metal, thus avoiding nickel skin allergy. It is also emphasized that due to their fixed design, the metal girders of the device described herein are removable/replaceable and can be reused, after sterilization, with any other patient, in the context of circular economy, something that to date has not been incorporated into any of the existing extraoral orthopedic devices with a similar purpose.

3. This device and the orthopedic device [27] follow the same practice: neither pushes the mandible backwards in order to move the maxilla forwards. In addition, this device consists of one connecting girder (3, FIG. 1) in the middle of the face instead of two on the sides, as in [27]. Because of that, the patient can also sleep on their side, which cannot be the case using the device [27].

4. Another advantage of this device in relation to [27] is that there is no need to have in its fully individualized skeletal support wreath a guided-adjustable-tightening mechanism because of its manufacturing process (scanning of the head—digital creation and 3D-printing of a wreath of exceptional accuracy). In this way, it is simpler, more comfortable and much more stable, because it is complemented by the elastic fixation straps (FIGS. 2, 4, 7). This prevents discomfort on the forehead and generally on the patient's head, which is vital for the patient's cooperation. This provides ease of application, stability and above all optimal comfort.

5. The direct protraction of the maxilla, mainly in patients who present opisthognathic maxilla in the face (maxillary retrognathia, maxillary deficiency), regardless of their Angle Class I or Class II dental relationship, is not referred to in the above-mentioned inventions of category E, except in [27]. In these malocclusions, the protracted maxilla can be held in its new position by this device and after that, the mandible can be moved forward by using another intraoral mechanism. Nevertheless, the mandible could also be protracted simultaneously by using a second mouth girder (4, FIG. 7) incorporated into the connecting girder (3), but inserted upside down. This is the only way the aetiologic therapy of the maxillary retrognathia in Angle Class I and II malocclusions can be achieved.

6. A further very significant disadvantage both of [27] and of all the above-mentioned devices and [28] of category E, except in [26], is the risk of dislodgment of the fully individualized skeletal support wreath, due to the upward and forward torque created during their use. Through the design of the herein described device this risk is minimized, because it is based on two key features: a. The methods used in the manufacture of the fully individualized skeletal support wreath, described below, ensure an excellent anatomical fit on the patient's head, something that is not provided in any of the above-mentioned devices in all categories, and which is necessary for its stable hold on the neurocranium, but also for the comfort of the patient when they wear something that is individualized made to their measurements. b. Due to the elastic fixation straps (7) that are attached to the fully individualized skeletal support wreath, the risk of its dislodgment is minimal, regardless of the magnitude of the forces aimed at the protraction of the maxilla or both jaws.

No matter how well the fully individualized skeletal support wreath fits, there are heads of patients with such anatomy (small area on the dorsal surface of the parietal bones and occipital bone) and/or patients with oily and fine hair, that no matter how fast the tightening mechanism [27] is in the frontal bone area, there is always the risk of dislodgment. This is due to the upward and forward torque of the dorsal portion of the fully individualized skeletal support wreath in response to the application of the force of elastic tractions to protract the maxilla. An opposite force must be exerted on the dorsal part of the wreath, which is achieved through the suspension of two elastic fixation straps (7, FIGS. 2, 4, 7) attached to this part. In this way, the wreath can be worn on any human skull and be used safely in the exercise of any force of direct traction of the maxilla and direct or indirect of the mandible, without the slightest risk of dislodgment.

The tightening mechanism in the frontal bone of [27] device, which is of vital importance, because of its excessive tightening, in order to avoid the dislodgment of the fully individualized skeletal support wreath, could create such pain in patients, that, in turn, drastically reduces the patient's motivation to cooperate.

7. Due to the methods of design and manufacture of the herein-presented fully individualized skeletal support wreath of cranial form, described below, which lead to an excellent anatomical fit and a precise placement of all its individual parts, it is manufactured finally with very high accuracy, which is not the case in any one of the existent devices [23-28].

8. Finally, as mentioned in the design of [27], the part of the fully individualized skeletal support wreath directly distal from the cylindrical slots, which accommodates the connecting cylindrical girders, “becomes extremely thin”, which, we found, could lead to a failure of the wreath in these areas, because these parts of the wreath receive an exceptional load during the operation of the device, proportional to the traction forces on the maxilla. The herein-described device boasts reinforcement webs (18), i.e. anti-bend supports, configured to withstand the actions applied during use.

Today, in patients with Angle Class II malocclusion, many of whom are in a growth phase and their growth potential can be modified, the orthodontist uses such techniques to move the permanent maxillary molars distally, aiming at an Angle Class I molar relationship (only dental and not skeletal—aetiological approach), ignoring very often the mandibular retrognathia. In the best cases, the orthodontist uses functional appliances, removable, fixed or hybrid types, to move the mandible forward. Even in these cases, the devices used are supported in the maxilla to reposition the mandible forward, which results in suppressing forward maxillary growth (when there is action, there is reaction).

In Angle Class Il malocclusion cases, in which the maxilla is located in a harmonic or in a retrognathic position within the face, which is also apparent from the increased nasolabial angle in the profile of the patient, the same therapy techniques are used, aiming at an Angle Class I dental relationship, ignoring the skeletal data of the jaws, as parts of the face in its entirety. In order to create harmonic and juvenile relationships of the two jaw bones within the face, orthofacial surgical interventions are used in adults, moving both the maxilla and the mandible forwards and secondarily creating an Angle Class I molar relationship [29].

In cases where the original location of the maxilla within the face is in a prognathic position (severe labial inclination of the upper permanent incisors, very reduced nasolabial angle) or in the true skeletal Class III cases, in which the mandible is prognathic because of its size with respect to the cranial base [30], we accept the use of the widely-used devices and techniques of today, for example in the true skeletal Class III cases, the compensatory support in the mandible and compression of the temporomandibular joints, in an effort to prevent the patient from orthognathic surgery in the future, but with possible negative implications.

The object of the invention is a convenient extraoral orthopedic mechanism, stable and precise in its adjustment and function, which in combination with an intraoral device is able to protract the maxilla directly, easily and without side-effects, as well as the mandible, directly or indirectly, mainly in cases of skeletal Class III and Class II malocclusions respectively. This mechanism can be used in cooperative, growing young patients.

Nowadays, in an Angle Class II malocclusion patient, it is impossible for an orthodontist to firstly reposition the maxilla further forward and deteriorate temporarily the dental Class II relationship, then reposition the mandible much further forward, holding simultaneously the maxilla in its protracted position, even with our suggested extraoral orthopedic device. Indeed, there is a plethora of growing young patients displaying an Angle Class II malocclusion, with both jaw bones in a retrognathic position whose facial growth can be modified. Nevertheless, the orthodontist “going with the flow” holds the maxilla in its original retrognathic location within the face and, with its support, tries to relocate the mandible forwards.

The proper head and body posture, unimpeded nasal breathing, mastication of hard as well as soft food and physiological swallow pattern should be very seriously considered, in the context of holistic orthodontic treatment.

This extraoral orthopedic device can be especially used in cooperative growing young patients, for whom the modification of the growth of their stomatognathic system is possible, for: a. The therapy of skeletal Class III malocclusion, protracting the maxilla directly after its “rapid expansion” [1-4, 8] without the simultaneous compression and impediment of mandible growth, thus avoiding: probable dysfunction of the TMJs, reduction of the tongue's vital space, which could lead, in turn, to possible sleep apnea and other problems. We especially mention the treatment of patients suffering from cleft lip and palate, in which the maxilla is retrognathic due to the connective tissue scars on the upper lip and upper jaw created by orthognathic/plastic surgery in the upper lip and maxilla. However, following the usual practice, the orthodontist uses a common physiologic mandible in size and position as support to protract a retrognathic maxilla [31]. Invariably in these cases, due to action-reaction, the mandible is moved backwards resulting in what has been explained above. b. The therapy of skeletal Class II malocclusion, with Angle Class I or II dental relationship, in the cases where the maxilla is retrognathic in its original position within the face. After the activation of viscerocranium sutures using the “rapid maxillary expansion” technique [1-4, 8] in the traditional way, the maxilla is protracted, initially, and subsequently held in its relocated position by the extraoral device. Finally, the mandible is directly or indirectly protracted using an additional intraoral mechanism. In this way, the growth modification of both maxilla and mandible gives an unmatched aesthetic result to the appearance of the whole face and greatly helps the function of breathing and tongue after the space in the mouth cavity has significantly increased. So, the probable cause of sleep apnea is notably reduced.

### DESCRIPTION OF DRAWINGS

The extraoral orthopedic device (1), as illustrated in detail in FIGS. 1-16, is basically composed of 4 parts: the fully individualized skeletal support wreath (2) on the neurocranium (9), the connecting girder (3), the mouth girder (4), the traction means, such as elastic bands (5, 10), through which the device is coupled with an intraoral mechanism of “rapid maxillary expansion” (6) or with another intraoral mechanism fixed in the mandible (11) and the elastic fixation straps (7), passing through the axillae with firm contact and around the shoulders reconnecting with their cranial ends, both of which pull and immobilize the wreath downwards, in order to avoid its dislodgment when the device (1) is in use.

FIG. 1 schematically illustrates: the frontal view of the device (1), the fully individualized skeletal support wreath (2) attached to the neurocranium (9), the connecting girder (3) in the middle of the face and the mouth girder (4). On the mouth girder are illustrated the elongated elements with a mounting means, such as the mounting screws (20), the anterior fixing screw (21) for the immobilization of the mouth girder and the threaded hole (22) for the posterior fixing screw (21) responsible also for the immobilization of the mouth girder. The markings (19) on the cranial and caudal parts of the connecting girder (3) are also illustrated.

FIG. 2 schematically depicts: the profile of the device (1) in use, the fully individualized skeletal support wreath (2) on the neurocranium (9), the connecting girder (3) with its markings (19), the mouth girder (4), the traction means (5), attached extraorally to the mounting screws (20) and intraorally to a mechanism of “rapid maxillary expansion” (6) and the right hand-side elastic fixation strap (7). The right elliptical slit (17) of the wreath for the passage of the cranial end of the elastic strap and its fixation area (26), as well as the cylindrical hole (14) in the wreath guiding the fixing screw (15) for the immobilization of the connecting girder (3) are also illustrated. After the straps (7) pass through the axillae and around the shoulders, their caudal ends are reconnected with their cranial ends through the adjustable and support strap buckles (27), either in the area of the chest, as represented here, or the back, depending on their insertion direction through the elliptical slits (17), in order to adjust the distance between the wreath and axillae, as well as their pulling power, which provides the required stability for the proper fixation of the wreath on the neurocranium and preventing the wreath (2) from being dislodged during operation of the device (1). The reinforcement webs (18), i.e. anti-bend supports, in the frontal and lateral areas of the wreath, are also presented.

In FIG. 3, the top view of the fully individualized skeletal support wreath (2) is illustrated. The metallic parts (13) create a cuboidal slot (12) responsible for the passage of the connecting girder (3). The fixing screws (15) for the immobilization of the connecting girder (3), the elliptical slits (17), the reinforcement webs (18), i.e. anti-bend supports, and the interior soft inlay (16), in the form of soft material pads, of the wreath (2), are also illustrated. The reinforcement webs (18) prevent the bending of the wreath (2) in its frontal and lateral areas during use of the device (1).

In FIG. 4, the back of a patient and the dorsal view of the device (1) with its fully individualized skeletal support wreath (2) placed on the neurocranium (9) are illustrated. The elliptical slits (17) for the passage of the cranial end of the elastic straps (7) and their attachments (26), the axillae (8), through which with firm contact and around the shoulders of the patient the straps (7) pass, are also shown. After the straps (7) pass through the axillae and around the shoulders, their caudal ends are reconnected with their cranial ends through the adjustable and support strap buckles (27). An additional connecting strap (65) adjoins the dorsal parts of the elastic straps (7).

In FIG. 5, the metallic connecting girder (3) of a rectangular cross-section, removeable and reusable after sterilization, and its markings (19) on the cranial part (28) of the connecting girder (3) responsible for an easy and accurate replacement and adjustment in its height in the fully individualized skeletal support wreath (2) and the markings (19) on the caudal part (30) of the connecting girder (3) responsible for an easy and accurate replacement and adjustment in the height of the mouth girder (4) along the connecting girder (3), as well as its angled middle part (29), are illustrated.

In FIG. 6, the aluminum-alloy mouth girder (4), which is removable and reusable after sterilization, is illustrated. In view A of this figure, the front surface of the mouth girder at a right angle, is shown, where the elongated elements with a mounting means, such as mounting screws (20), for the traction means, such as elastic bands (5, 10), the anterior fixing screw (21) for the immobilization of the mouth girder (4) and the threaded hole (22) for the posterior fixing screw (21) responsible also for the immobilization of the mouth girder are illustrated. In view B, the top view of the mouth girder (4) is shown. The separate aluminum-alloy part (24), incorporated into the mouth girder through the fixing screws (21) responsible for the immobilization of the mouth girder (4), as well as the cuboidal slot (25) for the passage of the connecting girder (3) are also illustrated. The threaded holes (23) responsible for the accommodation of the mounting screws (20) are also displayed. Finally, in view C, the rear view of the mouth girder at a right angle is shown. The mounting screws (20) for the traction means, the separate aluminum-alloy part (24), the posterior fixing screw (21) and the threaded hole (22) for the anterior fixing screw (21), responsible also for the immobilization of the mouth girder, are illustrated.

FIG. 7 schematically depicts: the profile of the device (1) in use, the fully individualized skeletal support wreath (2) on the neurocranium (9) with its reinforcement webs (18), the connecting girder (3) and two mouth girders (4), one for the direct traction of the maxilla and the other placed upside down for the direct traction of the mandible. The traction means, such as elastic bands (5, 10), attached extraorally to the mounting screws (20) and intraorally to a mechanism of “rapid maxillary expansion” (6) and another intraoral mechanism (11) fixed in the mandible correspondingly, as well as the right hand-side elastic fixation strap (7), are also illustrated.

In FIG. 8, a flowchart of the pre-scanning procedure of a person's head followed by scanning with a hybrid LED and infrared Light Source Handheld Color 3D scanner is presented.

In FIG. 9, in view A, the following six anatomical (craniometric) points are presented, on which self-adhesive dot-scannable stickers could be placed on the person's head before scanning: the Glabella (G), the left and right Orbitale (Or), the left and right Tragion (Trg) and the Labrale superior (Ls). In view B, the following three anatomical points are shown: the Inion (In, the outermost craniometric point of the external occipital protuberance) and the left and right outermost points of the prominences between the superior and inferior nuchal lines on the caudo-lateral areas of the occipital bone, the Sub-Inion left (SInL) and Sub-Inion right (SInR). The final two anatomical terms were named and abbreviated to facilitate communication. The nuchal lines of the occipital bone (supreme, I—superior, II—inferior, III), are also illustrated.

In FIG. 10, the rear view of the trunk and the head of a person wearing a wig cap, whose arms are raised and head is positioned in its natural head position (NHP), are illustrated. The left and right axillae (abbreviated as AxL and AxR), the SInL, the SInR, the Inion (In), as well as the apex of the patient's head (abbreviated as Ap) are also presented. The line section InAp passes normally through the midsagittal plane. The extensions of the line sections AxL-SInL and AxR-SInR create the angles L and R with the line crosses the anatomical (craniometric) points In and Ap. These two angles are used in the digital design of the two elliptical slits (17), when the applied elastic fixation straps (7) pass through the axillae (FIG. 4).

In FIG. 11, the rear view of the body and the head of a person wearing a wig cap, whose head is positioned in its natural head position (NHP), are illustrated. The left and right groins (abbreviated as GrL and GrR), the SInL, the SInR, the Inion (In), as well as the apex of the patient's head (Ap) are also shown. The line section InAp passes normally through the midsagittal plane. The extensions of the line sections GrL-SInL and GrR-SInR create the angles L′ and R′ with the line crosses the anatomical points In and Ap. These two angles are used in the digital design of the two elliptical slits (17), when the applied elastic fixation straps (7) pass through the groins (FIG. 12).

In FIG. 12, the rear view of the body of a patient wearing the extraoral orthopedic device (1) is depicted. The fully individualized skeletal support wreath (2) with its elliptical slits (17) for the passage of the cranial end of the elastic straps (7) and their attachments (26), the groins (31) of the patient, through which with firm contact and around the top of their thighs the straps (7) pass, are also illustrated. After the straps (7) pass through the groins and around the thighs, their caudal ends are reconnected with their cranial ends through the adjustable and support strap buckles (27) in the frontal area, as represented here, in order to adjust the distance between the wreath and the groin as well as their pulling power, which provides the required stability for the proper fixation of the wreath on the neurocranium and prevents the wreath (2) from being dislodged during operation of the device (1).

In FIG. 13, a flowchart of the workflow of the computer-aided design and manufacturing (CAD-CAM) of the fully individualized skeletal support wreath (2), also comprising the digital design of the caudal and cranial boundaries of the wreath using as references the midsagittal and Frankfort horizontal planes and at least the points, lines and linear segments (33-60), the individualized placement of the connecting girder (3), the positioning of the reinforcement webs (18) on the wreath (2) and the elliptical slits (17), as well as the offsetting responsible for the proper adaptation to the patient's hair thickness and the thickness and quality of the interior soft inlays, after the scanning of a person's head using a structured light 3D scanner, is illustrated.

In FIG. 14, the top view of the fully individualized skeletal support wreath (2) is illustrated. The only difference in comparison to FIG. 3 is the type of the interior soft inlay (16), which is an adjustable bag containing air, or air and a soft material, or liquid, or air and liquid. Air or liquid intake or outtake is performed through the valve (32) to such an amount in order to create a comfortable cushion covering the skull relief individually.

In FIG. 15, the references (points, lines and linear segments) used in the digital design of the caudal and cranial boundaries of the fully individualized skeletal support wreath (2) are presented.

The following craniometric points are defined on the editable surface of the patient's head and all of them are projected on the midsagittal plane: the Glabella (33); the Tragion (34); the Orbitale (35); the point (39) on the frontal bone contour located ten mm above Glabella; the Sub-Inion right (46); the Inion (49); the point (52) located on the frontal bone contour and 28 mm above the point 39; the apex (53) of the patient's head; the apex (58) of the curve located on the dorsal contour of the patient's head between the points 49 and 57; the point (57) located on the dorsal head contour, where the line 56 intersects this contour; the line (56) is parallel to Frankfort horizontal plane (36) and crosses the point 55; the point (55) located twenty mm below the craniometric point 53 on the line 54; the line (54) crosses the point 53 and is perpendicular to the Frankfort horizontal plane (36); the point (59) on the dorsal contour of the head located thirty mm above the point 58.

The following additional points are presented: the point (38), which is the middle of the concha (37) of the external ear on the Frankfort horizontal plane (36); the point (42), which is the point where the line 41 intersects the line 40 and it is the first control point of the spline curve (45); the line (41) perpendicular to the line 36 through the craniometric point 34; the line (40) crosses the craniometric point 39 and is parallel to 36; the second control point (44) of the spline curve (45) is located 2 mm more caudally to the point 42 and on the line 43; the line (43) is perpendicular to the line 36 through the point 38; the third control point (48) of the spline curve (45) is located on the line 47 and five mm above the point 46; the line (47) is perpendicular to the line 36 crossing the craniometric point 46; the fourth control point (50) of the spline curve (45) is located five mm above the point 49 and the last free point (51) of the spline curve (45) to relax tensions on the curve.

The spline curve (45) consisting of these four control points and one free point at its end is defined on the midsagittal plane. Its projected surface, which is perpendicular to the midsagittal plane, comes out to define the dorsal part of the caudal boundaries of the fully individualized skeletal support wreath (2) on the head surface of the patient. As with the spline curve (45), all the points, lines and linear segments are defined on the midsagittal plane. The perpendicular projection to the midsagittal plane of the linear segment 52-59 on the head surface represents the digital cranial boundaries of the wreath (2). The linear segment 39-42 is parallel to the Frankfort horizontal plane (36). Finally, the digital contour defined through the points, 39, 52, 59, 50, 48, 44, 42, 39, represents the projected profile contour of the digitally designed wreath (2).

In FIG. 16, the right and left maxillary halves (61) of a patient with a conventional mechanism of rapid maxillary expansion (6), banded on four maxillary teeth, are presented. On the lateral sides of the RME mechanism, the two welded metallic bars with their circular bends (62) at their frontal ends responsible for the attachment of the traction means (5), are also illustrated. In this traditional RME mechanism, two components on its frontal area are added. The connecting wires (63), between RME mechanism and acrylic pads (64), which comprise the frames for the acrylic pads (64), are illustrated. The acrylic pads (64) are located in very close contact with the palatal mucosa in the frontal region of the maxilla, right and left.

### Device Operation

Initially, “rapid maxillary expansion” using the traditional method is performed by any intraoral device [1-4, 8]. As an example, a modified “Hyrax” device (6, FIG. 16) banded to the maxillary posterior teeth by glass-ionomer cement is mentioned. In the buccal aspects of the bands, metallic bars, which have a circular bend (62) at their frontal ends, usually in the canine area, in order to be intraorally attached to the elastic bands, are welded. On the first permanent maxillary molars and on the second deciduous maxillary molars, when they are in the mouth, fixed bite-planes (posterior bite turbos) of a visible light-cure material, are placed. They are about 3 mm in height and they are used in order to impede maxillary molar extrusion, which is followed by downward and backward rotation of the mandible, by the opening of the midpalatal suture by the use of the “Hyrax” device. The bite planes are controlled on each visit to check for occlusal imprints of the mandibular teeth, which would obstruct forward maxillary movement and are repaired accordingly.

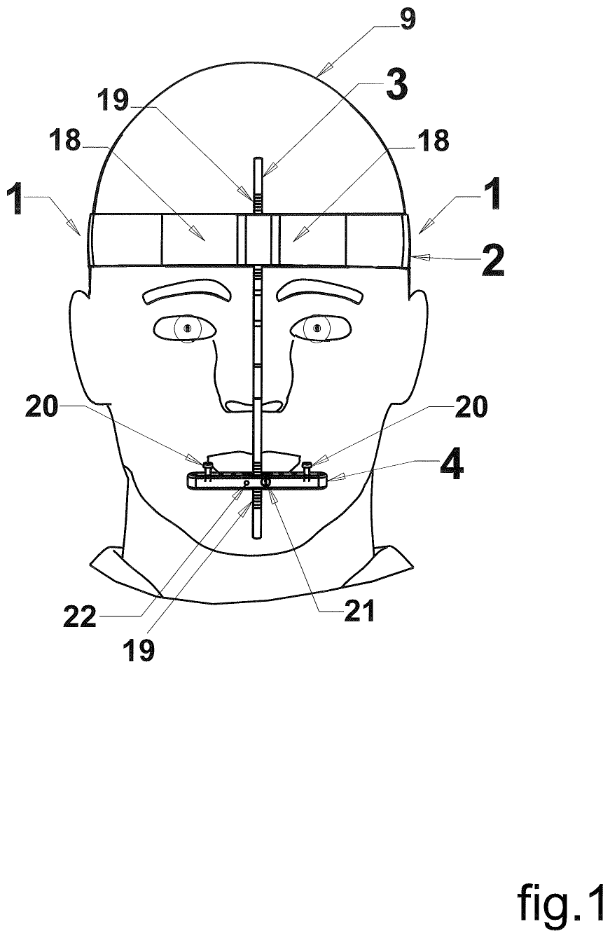

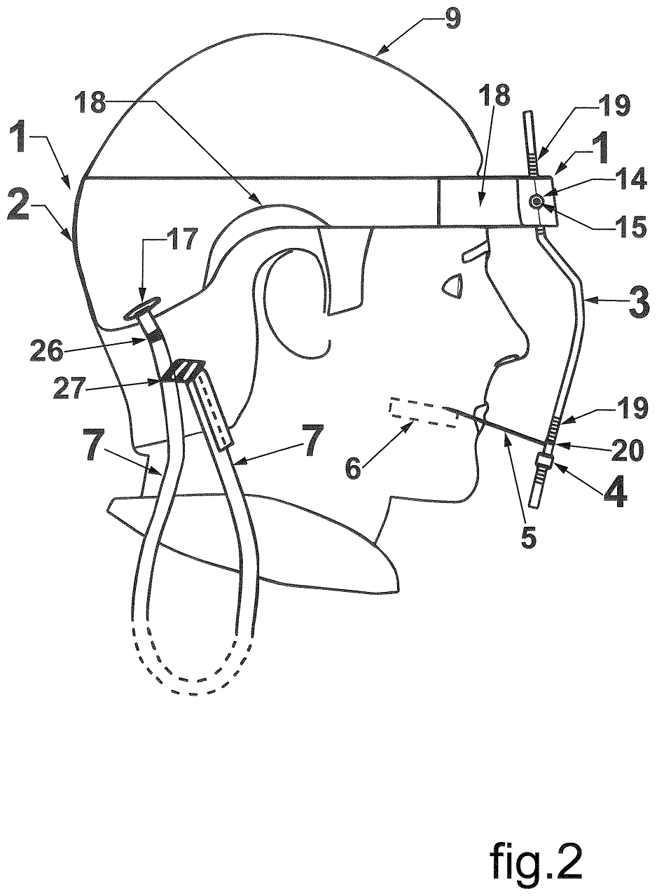

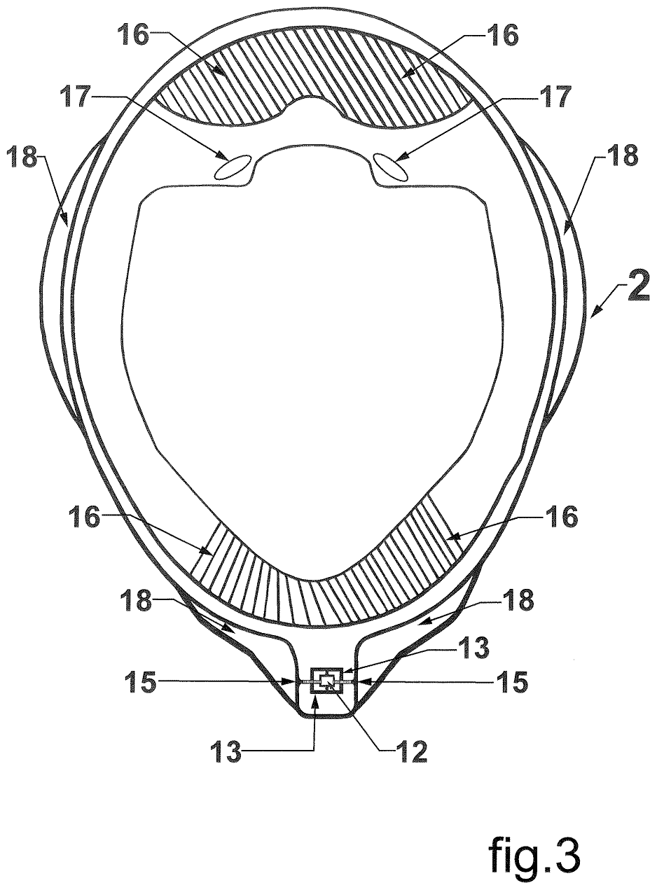

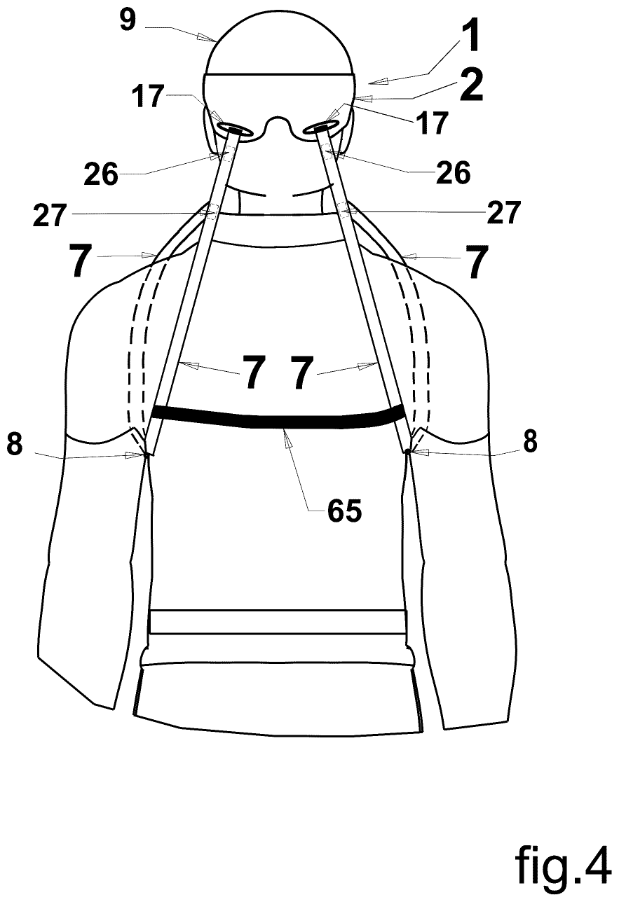

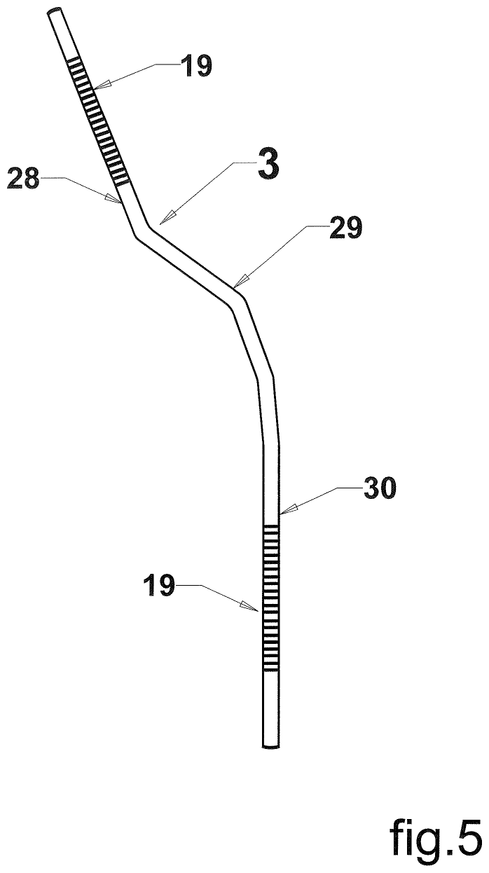

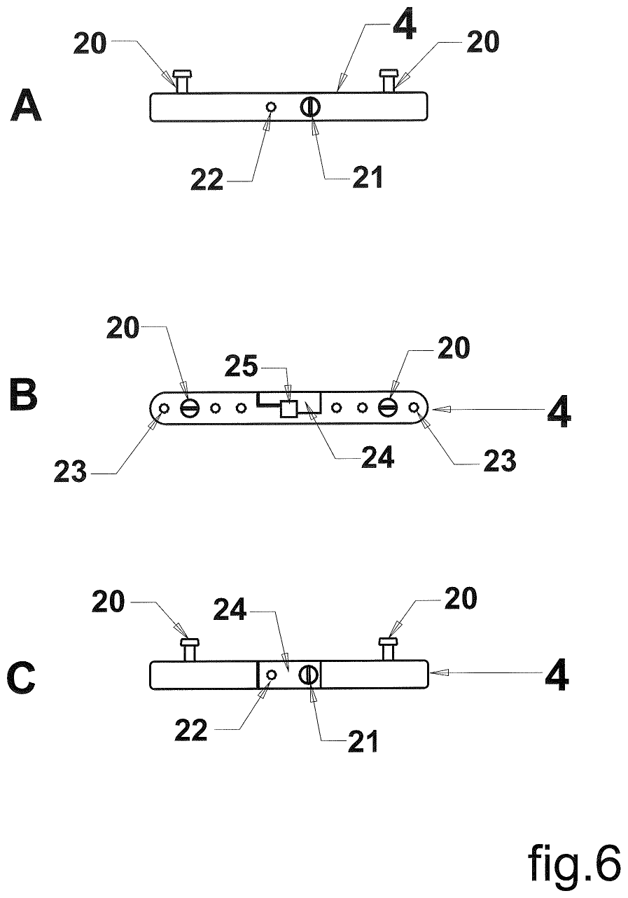

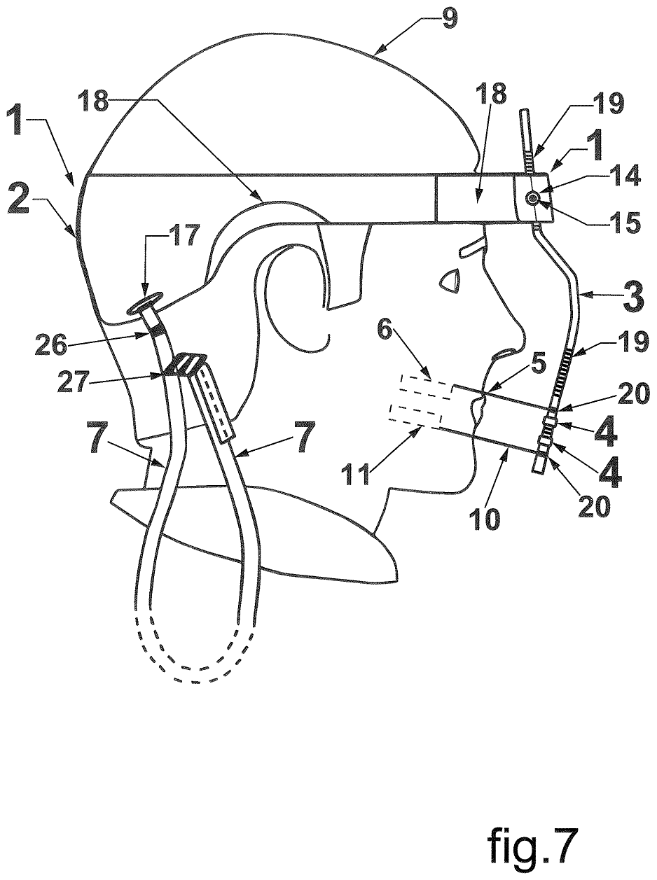

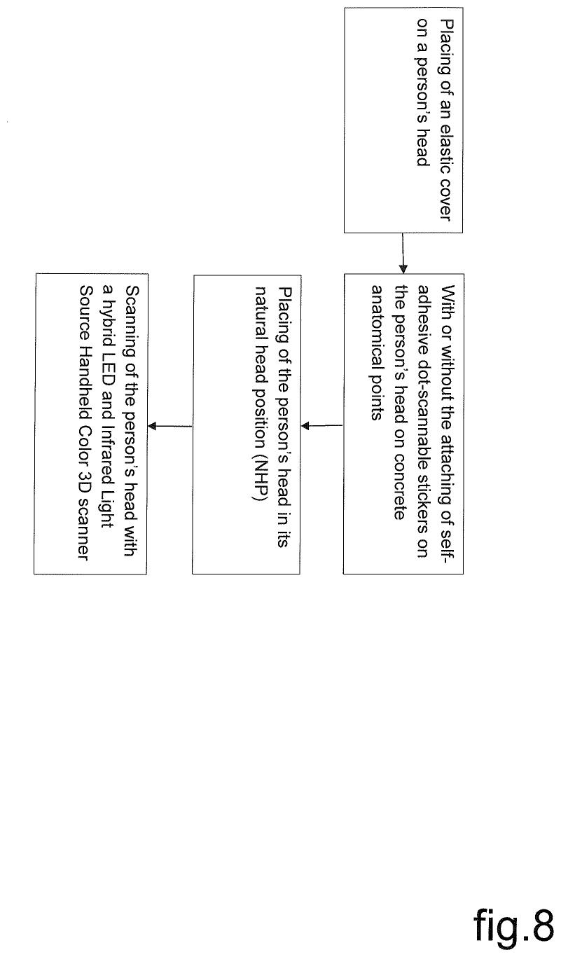

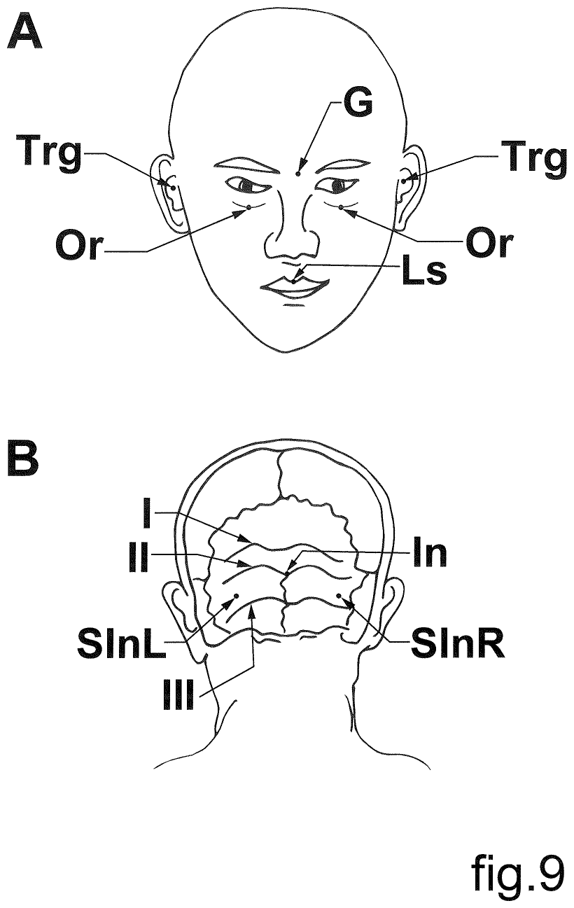

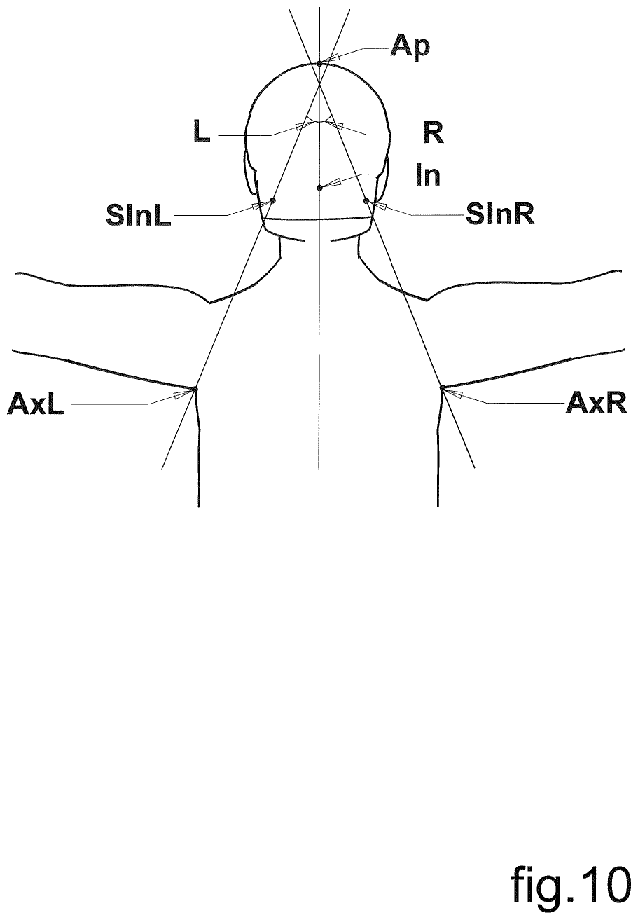

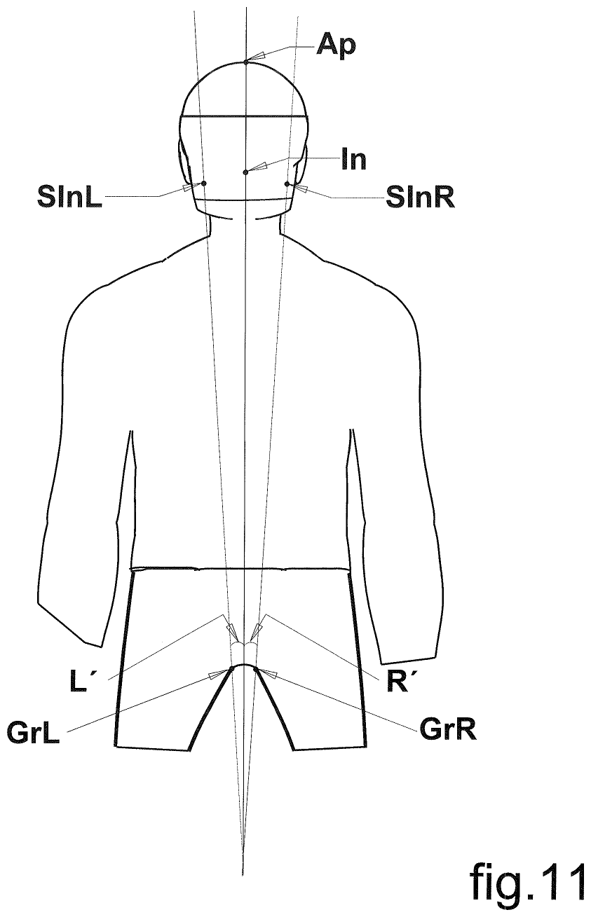

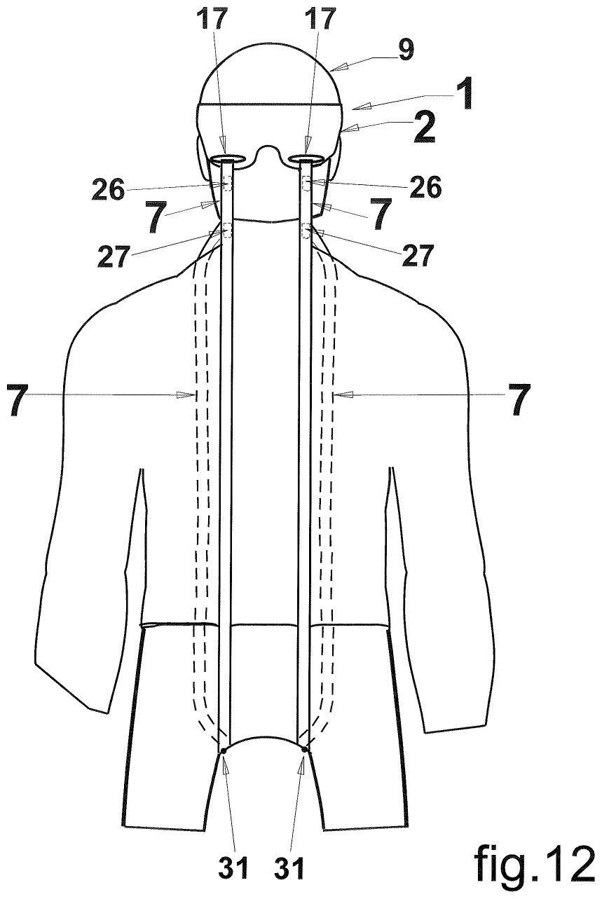

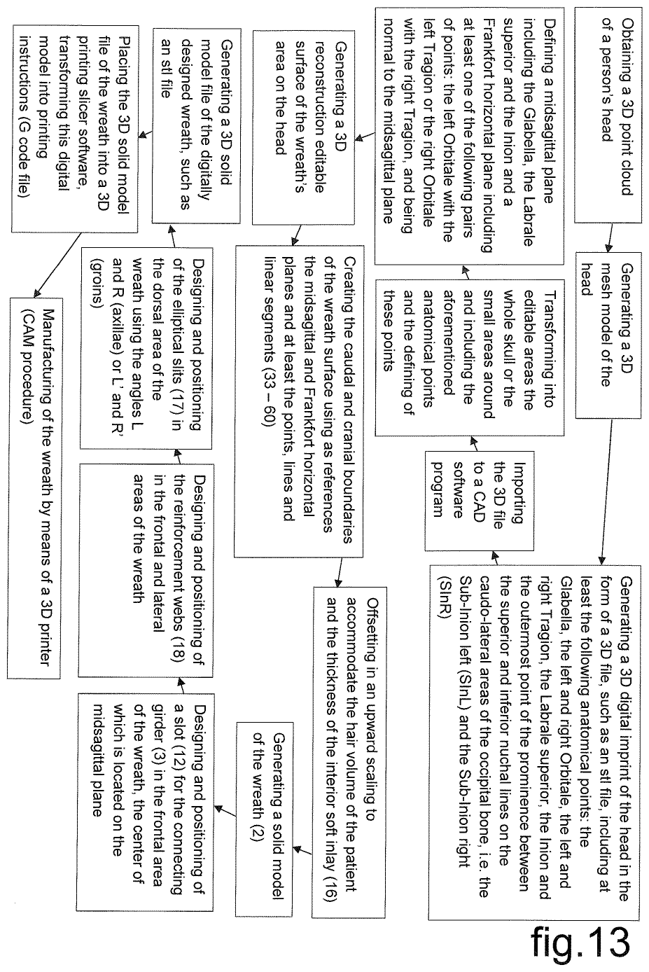

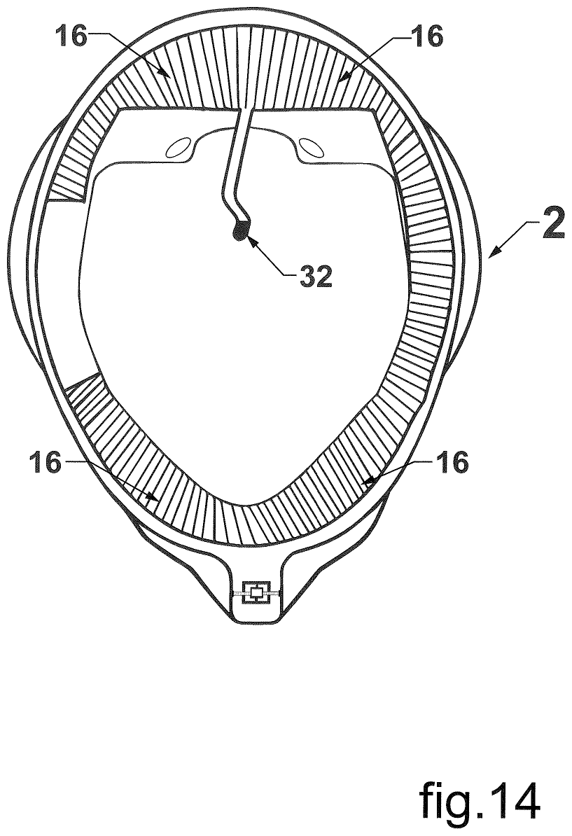

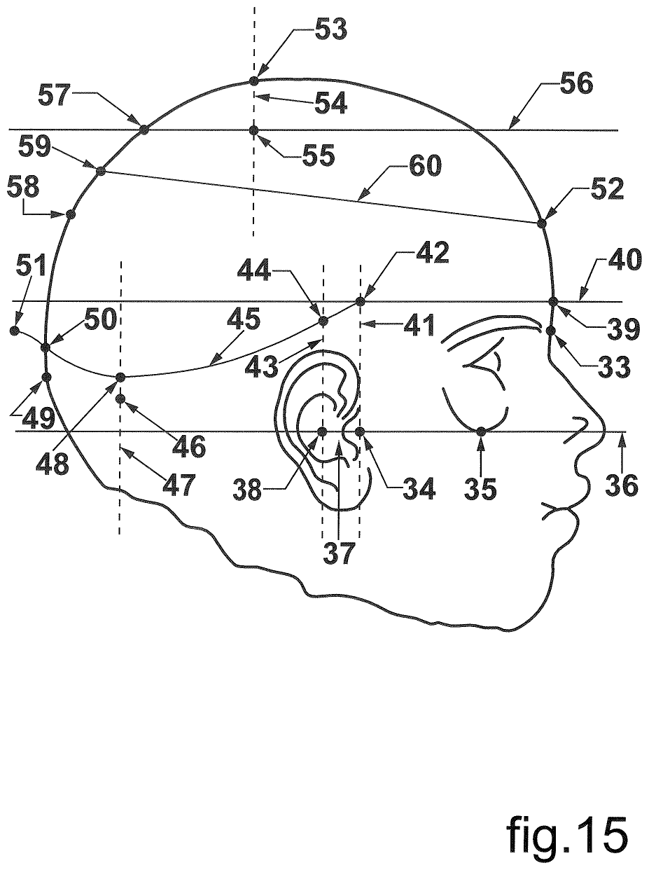

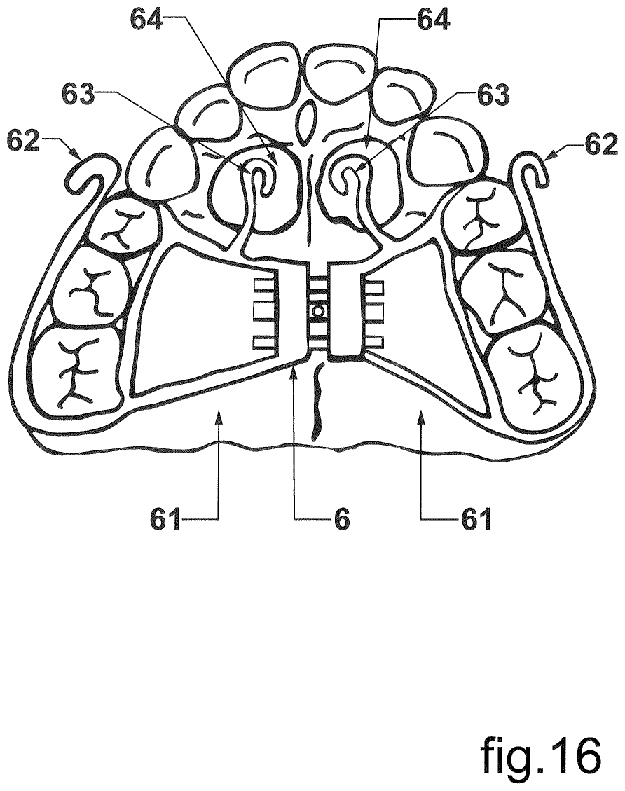

The extraoral orthopedic device is applied immediately after the disarticulation of the circumaxillary sutures aiming at an adequate maxillary protraction. After the end of the maxillary expansion and directly before the maxillary traction, two individualized acrylic pads (64), which had been manufactured on the palatal mucosa of the two maxillary halves in the front region of the maxilla, are placed at very close contact with the palatal mucosa and are connected with the two halves of the expansion screw by means of two additional wires (63), which had been supplemented on the conventional RME mechanism before its insertion. These two acrylic components (64), as additional anchorage, contribute to a “pure” maxillary traction preventing the maxillary support-teeth from being mesially moved because of the protractive forces, which reduces the orthopedic outcome and might result in an anterior crowding. These two acrylic segments (64) could also function as one unified component. First, the fully individualized skeletal support wreath is adjusted to the person's skull with the help of the interior soft inlay (16, FIGS. 3, 14) in the form of soft pads or an adjustable bag containing air, or air and soft material, or liquid, or liquid and air, producing maximum comfort and fit. Second, the connecting (3) girder of the device (1), after being inserted into the cuboidal slot (12, FIG. 3) of the fully individualized skeletal support wreath (2), is initially adjusted in height telescopically in relation to the patient's mouth slit and fixed by means of fixing screws (15, FIGS. 2, 3). Then, the mouth girder (4, FIGS. 1, 2, 6), after being inserted into the connecting girder (3) through its cuboidal slot (25), is also telescopically adjusted in height in order for the person's jaw to receive an optimal force direction for its traction. The immobilization in the connecting girder is achieved by means of the fixing screws (21, FIGS. 1, 6). Third, the fully individualized skeletal support wreath with the attached two girders is removed from the patient's head and the two elastic fixation straps (7) are connected with it through its elliptical slits (17) in their fixation areas (26). After that, the caudal ends of the straps coming through their buckles create a strap-loop in an anterior-posterior direction on each side of the wreath (FIGS. 2, 4, 7). Fourth, traction means, such as elastic bands (5, FIG. 2), are applied in order to protract the maxilla. Initially, the traction means are attached intraorally on the lateral hooks (62, FIG. 16) of the intraoral device (6, FIGS. 2 and 16). Then, the arms of the patient come through the inner side of the aforementioned strap-loops and the fully individualized skeletal support wreath (2, FIG. 1-4, 7) is primarily adapted (connecting girder in the middle of the face) and fixed to the patient's neurocranium (9, FIGS. 1, 2, 4, 7) through its perfect shape and its interior soft inlay (16, FIGS. 3, 14). Fifth, the two elastic fixation straps (7) are initially activated pulling the fully individualized skeletal support wreath downwards from its dorsal side. Then, the traction means (5) are attached extraorally to the two elongated elements with the mounting means, such as mounting screws (20, FIG. 2), of the mouth girder (4). Their pulling forces and their traction-angle are measured and are finally adjusted in relation to the patient's needs. If the protracting forces bring the connecting girder close to the patient's chin moving upwards and forwards the dorsal side of the wreath, the elastic fixation straps are additionally activated in such a way until an equilibrium occurs between the forces developed by the operation of the device (1) in its frontal and dorsal area, which is essential for its proper function. This last activation of the straps (7) is of vital importance because the risk of dislodgment during use of the device is virtually eliminated and additionally the distance between the mouth girder (4) and the patient's mouth slit remains unchanged (stable traction forces). In order to provide further stability, an extra connecting strap (65) adjoins the dorsal parts of the elastic fixation straps (7). This is in case the user raises their arms, especially during sleep. With the help of the connecting strap (65) the elastic fixation straps (7) maintain their original goal.

The extraoral attachment of the traction means is achieved ergonomically and symmetrically on the right and left side of the patient's head, also in respect to the patient's rima oris width, avoiding a trauma of the oral commissures, thanks to the various attachment positions (23, FIG. 6) on the mouth girder (4). The mandible could also be directly protracted using an additional mouth girder (4), placed upside down (traction means 10—intraoral mechanism fixed in the mandible 11, FIG. 7).

Summarizing, it is emphasized that this is an extraoral orthopedic device which can greatly help in the therapeutic modification of the maxillary and mandibular growth in skeletal Class III and Class II patients when it is especially used in cooperative, growing young individuals. Its main advantages are:

1. Because of its skeletal anchorage, primarily to the neurocranium and partly around the shoulders and through the axillae of each patient during maxillary protraction, there is no pressure on the mandible, thus avoiding any adverse effect on the temporomandibular joints arising from increased pressure during its use. Consequently, an enlargement of the oral cavity occurs with all the benefits that this implies.

2. Easy use of the device, not only in the therapy of skeletal Class III patients, but for the aetiologic therapy of skeletal Class II patients, even in the cases of Angle Class I or Class II malocclusions with retrognathic mandible but also retrognathic maxilla within the face, which, until now, is not the case by using extraoral or intraoral devices. Using this device, the mandible can also be protracted directly or indirectly. Particular mention of the use of this device is also made for operated cleft lip and palate patients, whose maxilla is often retrognathic due to the scars created by the operations on the upper lip and maxilla. These scars impede the physiologic growth of the maxilla, but the mandible usually has a physiologic position in the patient's skull.

3. Optimum symmetry in device settings when our device is used on the patient's skull in a versatile and safe manner. The applying traction means can be attached in the direction desired by the orthodontist both vertically and transversely, due to the ergonomic and practical design.

Initially, the fully individualized skeletal support wreath, based on the head scan, is manufactured. Additionally, the head perimeter of the patient is measured at the beginning of treatment. The thickness of the initial interior soft inlay, which partly covers the inner surface of the wreath, could be large enough in order that it can be replaced with a thinner interior soft inlay, in the case that within the therapy-period of time minor growth of the skull occurs. It becomes even easier in the case where the interior soft inlay is an adjustable bag containing air, or air and soft material, or liquid, or liquid and air. In this case, the thickness of the interior soft inlay can be reduced by removing air from the adjustable bag. On each visit, it is recommended that the head perimeter of the developing individual be measured, in order to regulate physiological enlargement of the human skull for each patient. If the skull growth, within this period of time, is significant, the wreath has to be replaced with a new, wider one adapted to the new skull dimensions.

Lastly, parts of this device could be manufactured with other materials. Indicatively, the carbon fiber material for the construction of the fully individualized skeletal support wreath is mentioned, which is extremely lightweight and durable. The wreath could also be manufactured from Nylon (PA11 or PA12). Other materials, such as PLA with similar properties to those of thermoplastic (ABS) could be used, but with a more ecological footprint (biodegradable and compostable). Both girders could consist of an aluminum-alloy or of a stainless steel-alloy or a combination of them, materials that can be reused after sterilization. Velcro type fasteners, clamping regulators between the wreath sections could also be integrated. Different thicknesses in the interior soft inlay, when it is offered only as a soft material pad and not as an adjustable air-and/or liquid-bag, depending on the hair volume of each individual or to adjust to the physiologic growth of the human skull, could be an additional service to each person.

The elastic fixation straps used for the proper fixation of the fully individualized skeletal support wreath and against its dislodgment would be able to pass, apart from through the axillae (FIGS. 2, 4, 7) and the groins, (FIG. 12), as already described, under the bottom of the feet (sole, plantar aspect). These are the three natural areas of the human body, that could offer the required stability for the wreath. The first mentioned body area (axilla) is closer to the wreath, more comfortable for the patient and more controlled.

### Method of Design and Manufacturing of the Fully Individualized Skeletal Support Wreath Using 3D Digital Data Acquisition Received After Scanning a Person's Head

The use of non-invasive methods, which would not harm the person's health, for the manufacture of a 3D printed customized facemask has previously been used [32]. In this way, the use of the relatively high dose of radiation involved in a CT or a CBCT [33-38] is avoided.

Cutting-edge technology at the current level of science, such as with a hybrid LED and Infrared Light Source Handheld Color 3D scanner is used in the workflow of an accurate creation of the fully individualized skeletal support wreath involved in the herein-described device. This is achieved either by digitizing the anatomy of the person's head, or by digitizing the geometry of the “wreath” created with impression material, as described below and with the help of reverse engineering.

An individualized wreath or headgear can be fabricated [39] with data acquired from a CT scan, which is an accurate but very invasive method especially when used at ages of growth.

### Preparation Before Scanning (FIGS. 8, 9)

a. An elastic cover, such as an elastic wig cap or an elastic swimming cap, is placed on the person's head. If the person has excessively thick hair, an elastic cap with a perforation on its top could be used, so that the greater volume of hair can be collected outside the elastic cap and kept away from the scanning areas where the fully individualized skeletal support wreath is placed.
b. Subsequently and before scanning, it may be useful through palpation to determine concrete anatomical points on the person's skull and to place self-adhesive dot-scannable stickers on the following nine anatomical (craniometric) points: the Glabella, the left and right Orbitale, the left and right Tragion, the Labrale superior, the Inion (the outermost craniometric point of the external occipital protuberance) and the left and right outermost points of the prominences between the superior and inferior nuchal lines on the caudo-lateral areas of the occipital bone [the Sub-Inion left (SInL) and Sub-Inion right (SInR)].
c. The person's head is placed in its natural head position (NHP), which implies that the head is in an upright posture, the eyes focused on a point in the distance at eye level, which indicates that the visual axis is horizontal.

### Digital Workflow (FIG. 13)

Next, the scanning of the person's head follows. After that, a 3D point cloud is obtained, which is generated into a 3D mesh model of the head. Then, this is generated into a 3D digital imprint of the head in the form of a 3D file, such as an stl file, including locating on a reference coordinate system the coordinates of a plurality of anatomical points of the subject involving at least the following points: the Glabella, the left and right Orbitale, the left and right Tragion, the Labrale superior, the Inion and the outermost points of the prominences between the superior and inferior nuchal lines on the caudo-lateral areas of the occipital bone, i.e. the Sub-Inion left and Sub-Inion right.

After that, the 3D file is imported to a CAD software program and the whole skull transformed into an editable area followed by the definition of the aforementioned anatomical points. If the computer capacity is not adequate, the small areas around and including the aforementioned anatomical points are transformed into editable areas and defined. Then, the definition of a midsagittal plane including the Glabella, the Labrale superior and the Inion and a Frankfort horizontal plane including at least one of the following pairs of points: the left Orbitale with the left Tragion or the right Orbitale with the right Tragion, and which is normal to the midsagittal plane, is carried out. Following this, the generation of a 3D reconstruction editable surface of the fully individualized skeletal support wreath's area on the head is performed, which is unnecessary in the case that the whole skull has already been transformed into an editable surface. Subsequently, the creation of the caudal and cranial boundaries of the wreath surface using as references the midsagittal and Frankfort horizontal planes, as well as at least the points, lines and linear segments (33-60) mentioned in the FIG. 15, is carried out.

Next, a general offsetting in an upward scaling to accommodate the hair volume of the person and the thickness of the interior soft inlay, in either form, is completed.

Then, a solid model of the fully individualized skeletal support wreath is generated.

The designing and positioning of the slot (12) for the universal connecting girder (3) in the frontal area of the fully individualized skeletal support wreath, the center of which is located on the midsagittal plane, follows.

The procedure continues with the designing and positioning of the reinforcement webs (18) in the frontal and lateral areas of the fully individualized skeletal support wreath.

After that, a negative offsetting in the frontal and dorsal area of the fully individualized skeletal support wreath on the areas which are going to accommodate the interior soft inlay, is performed in the case where the interior soft inlay is offered as soft material pads, but not for an adjustable bag containing air, air and soft material, liquid or air and liquid.

In the end, the designing and positioning of the elliptical slits (17) in the dorsal area of the fully individualized skeletal support wreath is carried out. The minor axes of these two elliptical slits pass through the line sections AxL-SInL and AxR-SInR (FIG. 10) in the case where the two elastic fixation straps (7) pass through the axillae and around the shoulders or through the line sections GrL-SInL and GrR-SInR (FIG. 11) in the case where the two elastic fixation straps (7) pass through the groins and around the thighs. The major axes of the two elliptical slits are perpendicular to their minor axes, as occurs in any ellipse. The lengths of the minor and the major axes of the elliptical slits are determined by the thickness and the width of the elastic fixation straps (7), correspondingly. The centers of the left and right elliptical slits are determined circa 25 mm above the SInL and SInR on the line sections AxL-SInL and AxR-SInR or GrL-SInL and GrR-SInR correspondingly, as well as the vertices (the points where the major axes cut the ellipses) and the lower co-vertices (the points where the minor axes cut the ellipses caudally) are at least 1 cm apart from the caudal boundaries of the wreath. The extensions of the line sections AxL-SInL and AxR-SInR create the angles Land R (FIG. 10) with the line crosses the anatomical points In and Ap. These two angles are used in the digital design of the two elliptical slits (17), when the applied elastic fixation straps (7) pass through the axillae (FIGS. 2, 4, 7). The extensions of the line sections GrL-SInL and GrR-SInR create the angles L′ and R′ (FIG. 11) with the line crosses the anatomical points In and Ap. These two angles are used in the digital design of the two elliptical slits (17), when the applied elastic fixation straps (7) pass through the groins (FIG. 12).

The next stage involves the generation of a 3D solid model file of the digitally designed fully individualized skeletal support wreath, such as an stl file (CAD workflow file).

After that, the 3D solid model file of the fully individualized skeletal support wreath is placed into a 3D printing slicer software, which transforms this digital model into printing instructions (G code file).

Finally, the fully individualized skeletal support wreath is manufactured by means of a 3D printer, CNC machines or robotics (CAM procedure).

The above-mentioned digital procedure can be followed using two modes of digital 3D data acquisition:

First, the fully individualized skeletal support wreath is roughly created directly on the skull within a traced contour by means of an impression material, such as polysiloxane (addition silicone type) or a thermoplastic material, such as those used in the manufacture of masks to immobilize the person's head, in particular during radiotherapy (radiotherapy immobilization devices). After the polymerization of the impression material, the inner surface of the “wreath”, which corresponds to the anatomy of the outer surface of the neurocranium, is scanned. In this way, the geometry of the neurocranium, created by means of the impression material, using a reverse engineering technique, is digitized.

Secondly, the best method, ergonomically and environmentally, for the CAD-CAM creation of the fully individualized skeletal support wreath is direct scanning of the person's head. Then, point processing and geometric model development is carried out using computer soft-ware and finally, it is printed using a 3D printer.

A computer program could be created comprising instructions which, when the program is executed by a computer, cause the computer to carry out certain steps of the above-mentioned method.

Its effectiveness in terms of the skeletal support of the invented extraoral orthopedic device and the comfort of the person during its application through this design, is superior, because it correlates with exceptional precision to the anatomy of the head of each person. It is no longer an ellipsoidal wreath, but a wreath of fully individualized cranial form, which corresponds perfectly to the anatomy of the person's head. Measurements by mechanical means, in our case, measurement with the 3D structured-light scanner, are not associated with any subjective errors. The prerequisite is of course that the digital impression instructions (data acquisition instructions), mentioned above, have been strictly followed, as well as the manufacturer's instructions, such as the correct setting (calibration) of the 3D scanner (scanning distance, room brightness, etc.). The only disadvantage of this technique is the need to process large information files, but this is easily overcome by using computers capable of processing such large 3D files.

### Other Applications

It can also be mentioned that this technique of digitally imaging human skulls with a 3D scanning camera using structured light and with the aid of the elastic cover, as described above, can be used to create protective helmets, which can be used in the context of various activities (sports, workplaces, etc.) where a head cover is deemed necessary for protection and safety reasons. A second application of this method could be to create individualized symmetrical helmets worn especially during the first year of age of individuals presenting asymmetries in their skull morphology as symmetry guidance. Skull asymmetries could be corrected also employing non-invasive methods for the manufacture of helmets using modelling putty spacer material [40].

### REFERENCES

1. Haas A J. Rapid expansion of the maxillary dental arch and nasal cavity by opening the midpalatal suture. Angle Orthod. 1961;31(2):73-90.
2. Biederman W. A hygienic appliance for rapid expansion. J Pract Orthod. 1968;2(2):67-70.
3. Haas A J. Palatal expansion: Just the beginning of dentofacial orthopedics. Am J Orthod. 1970;57(3):219-55.
4. McNamara J A. An Orthopedic Approach to the Treatment of Class III Malocclusion in Young Patients. J Clin Orthod. 1987;21(9):598-608.
5. Liou E J, Tsai W C. A new protocol for maxillary protraction in cleft patients: repetitive weekly protocol of alternate rapid maxillary expansions and constrictions. Cleft Palate-Craniofacial Journal, 2005;42:121-27.
6. Canturk B H, Celikoglu M. Comparison of the effects of face mask treatment started simultaneously and after the completion of the alternate rapid maxillary expansion and constriction procedure. Angle Orthod. 2015;85(2):284-91.
7. Liu W, Zhou Y, Wang X, Liu D, Zhou S. Effect of maxillary protraction with alternating rapid palatal expansion and constriction vs expansion alone in maxillary retrusive patients: A single-center randomized controlled trial. Am J Orthod Dentofacial Orthop. 2015;148(4):641-51.
8. Wilmes B, Nienkemper M, Ludwig B, Kau C H, Drescher D. Early Class III Treatment with a Hybrid-Mentoplate Combination. J Clin. Orthod. 2011;45(1):1-7.
9. Delaire J. Confection du masque orthopedique. Rev Stomatol. 1971;72(5):579-84.
10. Delaire V J, Verdon P, Floor J. Ziele und Ergebnisse extraoraler Züge in postero-anteriorer Richtung in Anwendung einer orthopädischen Maske bei der Behandlung von Fällen der Klasse III. Fortschr Kiefer Orthop 1976;37:246-262.
11. Petit H. Adaptations following accelerated facial mask therapy in clinical alteration of the growth face. In: McNamara J A Jr, Ribbens K A, Howe R P (eds). Clinical Alteration of the Growing Face, monograph 14, Craniofacial Growth Series. Ann Arbor, MI: University of Michigan, 1983.
12. Turley P K. Orthopedic correction of Class III malocclusion with palatal expansion and custom protraction headgear. J Clin Orthod. 1988;22(5):314-325.
13. https://www.orthazone.com/Face-Mask-ReversePull-Headgear-T%C3%BCbinger-Model-dtu-109
14. https://www.google.com/search?client=firefox-b-d&q=sky+hook+head+gear
15. Bisnoff H. L. Fracture Appliance. Jul. 27, 1943; U.S. Pat. No. 2,325,300
16. Hegmann M, Rüther A K. The Grummons face mask as an early treatment modality within a class III therapy concept. J Orofacial Orthop. 2003;64(6):450-6.
17. https://www.leone.it/english/services/download/Cat_Orthodontic_Eng.pdf
18. Alcan T, Keles A, Erverdi N. The effects of a modified protraction headgear on maxilla. Am J Orthod Dentofacial Orthop. 2000;117:27-38.
19. De Clerck H J, Cornelis M A, Cevidanes L H, Heymann G C, Tulloch C J. Orthopedic traction of the maxilla with miniplates: a new perspective for treatment of midface deficiency. J Oral Maxillofacial Surg. 2009;67(10):2123-9.
20. Yatabe M, Garib D, Faco R, de Clerck H, Souki B, Janson G, Nguyen T, Cevidanes L, Ruellas A. Mandibular and glenoid fossa changes after bone-anchored maxillary protraction therapy in patients with UCLP: A 3-D preliminary assessment. Angle Orthod. 2017 May;87(3):423-431.
21. Proffit, William, R. et al. Treatment of Skeletal Transverse and Class III Problems (chapter 13). Contemporary Orthodontics (6th Edition). Elsevier—OHCE, 2018.
22. Cha B K, Choi D S, Ngan P, Jost-Brinkmann P G, Kim S M, Jang Is. Maxillary protraction with miniplates providing skeletal anchorage in a growing Class III patient. Am J Orthod Dentofacial Orthop. 2011;139:99-112.
23. Pourcho W S. Orthodontic appliance. U.S. Pat. No.: 5,158,451, Oct. 27, 1992.
24. Ngiam Ioachim. Mandibular advancement. U.S. Pat. No.: 0,040,301, Feb. 16, 2012.
25. DEL GOBBO WANDA. ORTHOPEDIC DEVICE FOR THE PROTRACTION OF THE MAXILLARY ARC. CPC: A61C7/06, IPC: A61C7/06, Publication info: WO2017089971 (A1) 2017 Jun. 1.
26. KAHN SANDRA VIVIAN, FLORES CERCEDA MABEL, KAHN LEVENTHAL ILAN. MAXILLARY PROTRACTION DEVICE. CPC: A61B2560/0475, A61B5/0008, A61B5/01, IPC: A61B5/00, A61B5/01, A61C7/06, Publication info: US2018028282 (A1), 2018 Feb. 1; US10166089 (B2) 2019 Jan. 1.
27. KOUTZOGLOU STYLIANOS, KOUTZOGLOU ELENI. EXTRAORAL ORTHOPEDIC DEVICE FOR THE DIRECT PROTRACTION OF THE MAXILLA AND THE INDIRECT PROTRACTION OF THE MANDIBLE. IPC: A61C7/06; A61C7/36; A61F5/56; CPC: A61C7/06 (EP, GR); A61C7/36 (GR); A61F5/05891 (EP); A61F5/56 (EP, GR); A61C7/36 (EP); Publication info: WO2021090035A1 Doi:https://worldwide.espacenet.com/patent/search/family/071120204/publication/WO20 21090035A1?q=WO%202021%2F090035
28. GREENE et al., EXTERNAL FIXATION DEVICE FOR CRANIAL MAXILLOFACIAL DISTRACTION. US PATENT 2004/0199094 A1
29. Lorente C, Alfaro F H, Vela M P, Lorente P, Lorente T. Surgical-Orthodontic approach for facial rejuvenation based on a reverse facelift. Progress in Orthodontics (2019) 20:34 https://doi.org/10.1186/s40510-019-0287-8
30. Schwarz A. M.: Die Röntgenostatik. Urban & Schwarzenberg, Wien-Innsbruck 1958.
31. Yatabe M., Garib D. G., André de Souza Faco R, de Clerck H., Janson G, Nguyen T., Lucia Helena Soares Cevidanes L. H. S., Ruellas A. C. Bone-anchored maxillary protraction therapy in patients with unilateral complete cleft lip and palate: 3-dimensional assessment of maxillary EFFECTS. AM J ORTHOD DENTOFACIAL ORTHOP. 2017 September;152(3):327-335 DOI: 10.1016/j.ajodo.2016.12.024
32. Franchi L, Vichi A, Marti P, Lampus F, Guercio S, Recupero A, Giuntini V, Goracci C. 3D Printed Customized Facemask for Maxillary Protraction in the Early Treatment of a Class III Malocclusion: Proof-of-Concept Clinical Case. Materials 2022, 15(11), 3747; https://doi.org/10.3390/ma15113747
33. Hans M G, Palomo J M, Valiathan M. History of imaging in orthodontics from Broadbent to cone-beam computed tomography. Am J Orthod Dentofacial Orthop. 2015;148:914-21. DOI:https://doi.org/10.1016/j.ajodo.2015.09.007
34. Velasco Regina Casian Ruiz, Liang Hui Cone-Beam Computed Tomography (CBCT) Applications in Dentistry. DOI: https://www.dentalcare.com/en-us/professional-education/ce-courses/ce531/overview
35. Yeh J K., Chen C H. Estimated radiation risk of cancer from dental cone-beam computed tomography imaging in orthodontics patients. BMC Oral Health 18, 131 (2018). DOI:https://doi.org/10.1186/s12903-018-0592-5
36. Dental Cone-beam Computed Tomography https://www.fda.gov/radiation-emitting-products/medical-x-ray-imaging/dental-cone-beam-computed-tomography #benefitsrisks
37. Safety and Efficacy of a New and Emerging Dental X-ray Modality. https://cordis.europa.eu/project/id/212246
38. Pediatric X-ray Imaging https://www.fda.gov/radiation-emitting-products/medical-imaging/pediatric-x-ray-imaging
39. PARK M R. HEADGEAR FOR FIXING MOUTHPIECE. US PATENT 2021/0045844 A1
40. GORDON et al. IN SITU MOLDED ORTHOTIC AND METHOD FOR ITS FABRICATION. US PATENT 2012/0296249 A1

### TABLE OF REFERENCE SIGNS OF DRAWINGS

1: The extraoral orthopedic device for the protraction of the human jaws
2: The fully individualized skeletal support wreath
3: The connecting girder placed in the middle of the fully individualized skeletal support wreath on its frontal area
4: The mouth girder
5: The traction means, such as elastic bands, attached extraorally to the elongated elements with their mounting means, such as mounting screws (20), and intraorally to the mechanism of “rapid maxillary expansion” (6) in the maxilla.
6: The intraoral mechanism fixed in the maxilla
7: The elastic fixation straps of the fully individualized skeletal support wreath
8: The axilla
9: The person's neurocranium
10: The traction means, such as elastic bands, attached extraorally to the elongated elements with their mounting means, such as mounting screws (20), and intraorally to a mechanism (11) fixed in the mandible.
11: The intraoral mechanism fixed in the mandible
12: The cuboidal slot in the frontal area of the fully individualized skeletal support wreath (2) responsible for the passage of the connecting girder (3).
13: The metallic parts which create the cuboidal slot (12) in the fully individualized skeletal support wreath.
14: The cylindrical hole in the fully individualized skeletal support wreath guiding the fixing screw (15)
15: The fixing screw for the immobilization of the connecting girder (3)
16: The interior soft inlay of the fully individualized skeletal support wreath (2) in the form of a soft material pad or an adjustable bag containing air, or air and a soft material, or liquid, or air and liquid
17: The elliptical slits in the dorsal area of the fully individualized skeletal support wreath for the passage of the cranial end of the elastic fixation straps (7)
18: The reinforcement webs, or anti-bend supports, to prevent bending of the fully individualized skeletal support wreath (2) in its frontal and lateral areas during use of the device (1)
19: The markings on the cranial part (28) of the connecting girder (3) responsible for an easy and accurate replacement and adjustment in its height in the fully individualized skeletal support wreath (2) and the markings on the caudal part (30) of the connecting girder (3) responsible for an easy and accurate replacement and adjustment in the height of the mouth girder (4) along the connecting girder (3).
20: The elongated elements with a mounting means, such as mounting screws on the mouth girder (4), responsible for the attachment of the traction means (5 or 10), such as elastic bands.
21: The fixing screws for the immobilization of the mouth girder (4)
22: The threaded holes for the anterior and posterior fixing screws (21) responsible for the immobilization of the mouth girder (4).
23: The threaded holes responsible for the accommodation of the elongated elements with mounting means (20).
24: The separate aluminum-alloy part incorporated into the mouth girder (4) through the fixing screws (21) responsible for the immobilization of this girder.
25: The cuboidal slot for the passage of the connecting girder (3)
26: The attachment of the cranial end of the elastic fixation straps (7)
27: The buckle responsible for the adjustment of the elastic fixation straps in height and to their pulling-power.
28: The cranial part of the connecting girder (3)
29: The middle part of the connecting girder (3)
30: The caudal part of the connecting girder (3)
31: The groin
32: The valve of the adjustable bag (16) for the individualized air or liquid intake or outtake
33: The craniometric point Glabella (G)
34: The craniometric point Tragion (Trg)
35: The craniometric point Orbitale (Or)
36: The Frankfort horizontal plane
37: The concha of external ear
38: The middle of the concha on the Frankfort horizontal plane
39: The craniometric point on the frontal bone contour located ten mm above Glabella
40: The line crossing the craniometric point 39 and parallel to 36
41: Perpendicular to the Frankfort horizontal plane through the craniometric point 34
42: The point where line 41 intersects line 40 and is the first control point of the spline curve 45.
43: Perpendicular to the Frankfort horizontal plane through the point 38
44: The second control point of the spline curve 45, located two mm more caudally to the point 42 and on the line 43
45: The spline curve consisting of four control points and one free point at its end
46: The craniometric point Sub-inion right (SInR)
47: Perpendicular to Frankfort horizontal plane crossing the craniometric point 46
48: The third control point of the spline curve 45, which is located on the perpendicular 47 and five mm above the craniometric point 46.
49: The craniometric point Inion (In)
50: The fourth control point of the spline curve 45, which is located five mm above the craniometric point 49 on the dorsal contour of the head.
51: The free point of the spline curve at its end
52: The craniometric point located on the frontal bone contour twenty-eight mm above the craniometric point 39.

53: The apex of the patient's head
54: The perpendicular to Frankfort horizontal plane crossing the craniometric point 53
55: The point on the line 54 located twenty mm below the craniometric point 53.
56: The line parallel to Frankfort horizontal plane crossing the point 55
57: The craniometric point, which is located on the dorsal contour of the patient's head, where the line 56 intersects the head contour. 58: The apex of the curve of the patient's head between the craniometric points 49 and 57
59: The craniometric point on the dorsal contour of the head located thirty mm above the craniometric point 58
60: The linear segment 52-59 representing the digital cranial boundaries of the fully individualized skeletal support wreath (2)
61: The right and left halves of the maxilla
62: The right and left circular bends on the welded metallic bars responsible for the attachment of the traction means (5).
63: The connecting wires between RME and acrylic pads (64)
64: The right and left acrylic pads on the frontal palatal mucosa right and left sections correspondingly
65: The connecting strap between the elastic fixation straps (7)

1-25. (canceled)

26. An extraoral orthopedic device, comprising:
a fully individualized skeletal support wreath with a frontal area and a dorsal area opposite the frontal area, whereby the fully individualized skeletal support wreath is configured to be placed on a periphery of a neurocranium of a user;
two elastic fixation straps having support and adjustment means configured to attach the two elastic fixation straps to complementary support means provided on the dorsal area of the fully individualized skeletal support wreath and around the shoulders and through the axillae or through the groins of the user, to adjust a length and a pulling-power of the straps between the wreath and the axillae or groins during use;
a mouth girder provided with means to attach at least one traction means to an intraoral mechanism;
a connecting girder connecting the fully individualized skeletal support wreath with the mouth girder;
connecting and adjustment means configured to rigidly connect the connecting girder at the frontal area of the fully individualized skeletal support wreath at a location along its cranial part;
additional connecting and adjustment means disposed on said mouth girder;
wherein the connecting and adjustment means and the additional connecting and adjustment means are configured to rigidly connect the mouth girder with the connecting girder, and the connecting girder with the fully individualized skeletal support wreath, to adjust a relative position of the mouth girder along a caudal part of the connecting girder, and the connecting girder with the fully individualized skeletal support wreath, so as to control a direction of the traction means during use.

27. The extraoral orthopedic device according to claim 26, further comprising a connecting strap between the two elastic fixation straps.

28. The extraoral orthopedic device according to claim 26, wherein the fully individualized skeletal support wreath is of cranial form.

29. The extraoral orthopedic device according to claim 26, wherein the connecting girder has a universal design of a profile of a human face and contains a cranial part disposed at an angle with a middle part, the middle part being disposed at an angle with a caudal part.

30. The extraoral orthopedic device according to claim 26, wherein the connecting girder and the mouth girder are made of a material based on, or an alloy, including any one or more of the following materials: a) titanium, b) carbon steel, c) alloy steel d) stainless steel, e) aluminum alloy, f) carbon fiber, g) polymer.

31. The extraoral orthopedic device according to claim 26, wherein the fully individualized skeletal support wreath is one of:
a continuous rigid and solid ring with non-adjustable dimensions;
a continuous rigid and solid ring with an interior soft inlay;
formed as a soft material pad;
formed as an adjustable airbag inflated or deflated using a valve;
formed as an adjustable bag containing air and a soft material, or air and a liquid, that is inflated or deflated using a valve.

32. The extraoral orthopedic device according to claim 26, wherein the fully individualized skeletal support wreath further includes reinforcement webs configured to withstand actions applied by the user, and wherein the reinforcement webs extend along an arc on the periphery of the fully individualized skeletal support wreath.

33. The extraoral orthopedic device according to claim 26, wherein a single connecting girder is disposed between the fully individualized skeletal support wreath and the mouth girder.

34. The extraoral orthopedic device according to claim 26, wherein the mouth girder includes at least one elongated element with a mounting means extended laterally from the mouth girder and configured to attach traction means.

35. The extraoral orthopedic device according to claim 34, wherein the at least one elongated element is configured to point towards the cranial and/or the caudal part of the mouth girder.

36. The extraoral orthopedic device according to claim 26, wherein the mouth girder has an orthogonal cross section.

37. The extraoral orthopedic device according to claim 26, further comprising a second mouth girder that is connected to the connecting girder, wherein the mouth girder and the second mouth girder are generally disposed in parallel.

38. The extraoral orthopedic device according to claim 26, wherein the at least one traction means is connected to an intraoral maxillary mechanism configured to withstand actions applied on the maxillary teeth.

39. The extraoral orthopedic device according to claim 26, wherein at least the connecting girder and the mouth girder are removable, independent elements.

40. A computer-implemented method of manufacturing a fully individualized skeletal support wreath, comprising:
obtaining a 3D point cloud of a head;
generating a 3D mesh model of the head;
generating a 3D digital imprint of the head in the form of a 3D file, including locating, on a reference coordinate system, coordinates of a plurality of anatomical points of a subject including at least: the Glabella, the left and right Orbitale, the left and right Tragion, the Labrale superior, the Inion, and outermost points of prominences between the superior and inferior nuchal lines on the caudo-lateral areas of the occipital bone;
importing the 3D file to a CAD software program;
transforming into editable areas the whole head or at least the small areas around and containing the aforementioned anatomical points and the defining of these points;
defining a midsagittal plane including the Glabella, the Labrale superior and the Inion and a Frankfort horizontal plane including at least one of the following pairs of points: the left Orbitale with the left Tragion, or the right Orbitale with the right Tragion, and being normal to the midsagittal plane;
generating a 3D reconstruction editable surface of at least part of the fully individualized skeletal support wreath's area on the head of the user;
creating caudal and cranial boundaries of the fully individualized skeletal support wreath surface using as references the midsagittal and Frankfort horizontal planes and at least the points, lines and linear segments labelled as parameters 33-60, and defined as:
parameter 33 is the craniometric point Glabella;
parameter 34 is the craniometric point Tragion;
parameter 35 is the craniometric point Orbitale;
parameter 36 is the Frankfort horizontal plane;
parameter 37 is the concha of external ear.
parameter 38 is the middle of the concha on the Frankfort horizontal plane;
parameter 39 is the craniometric point on the frontal bone contour located five to twenty mm above Glabella;
parameter 40 is the line crossing the craniometric point (parameter 39) and parallel to parameter 36;
parameter 41 is the perpendicular to the Frankfort horizontal plane through the craniometric point (parameter 34);
parameter 42 is the point where line of parameter 41 intersects line of parameter 40 and is the first control point of the spline curve of parameter 45;
parameter 43 is the perpendicular to the Frankfort horizontal plane through the point of parameter 38;
parameter 44 is the second control point of the spline curve of parameter 45, located at least one mm more caudally to the point of parameter 42 and on the line of parameter 43;
parameter 45 is the spline curve consisting of at least four control points and one free point at its end;
parameter 46 is the craniometric point Sub-inion right;
parameter 47 is the perpendicular to Frankfort horizontal plane crossing the craniometric point of parameter 46;
parameter 48 is the third control point of the spline curve of parameter 45, which is located on the perpendicular of parameter 47 and at least one mm above the craniometric point of parameter 46;
parameter 49 is the craniometric point Inion;
parameter 50 is the fourth control point of the spline curve of parameter 45, which is located at least one mm above the craniometric point of parameter 49 on the dorsal contour of the head;
parameter 51 is the free point of the spline curve of parameter 45 at its end;
parameter 52 is the craniometric point located on the frontal bone contour at least twenty mm above the craniometric point of parameter 39;
parameter 53 is the apex of the user's head;
parameter 54 is the perpendicular to Frankfort horizontal plane crossing the craniometric point of parameter 53;
parameter 55 is the point on the line of parameter 54, which is located on the craniometric point of parameter 53 or at least one mm below the craniometric point of parameter 53;
parameter 56 is the line parallel to Frankfort horizontal plane crossing the point of parameter 55;
parameter 57 is the craniometric point, which is located on the craniometric point of parameter 53 or on the dorsal contour of the user's head, where the line of parameter 56 intersects the head contour;
parameter 58 is the apex of the curve of the user's head between the craniometric points of parameters 49 and 57;
parameter 59 is the craniometric point, which is located on the craniometric point of parameter 53 or on the dorsal contour of the head located at least one mm above the craniometric point of parameter 58;
parameter 60 is a linear segment between parameters 52-59 representing the digital cranial boundaries of the fully individualized skeletal support wreath;
offsetting in an upward scaling to accommodate the hair volume of the user and the thickness of the interior soft inlay of the fully individualized skeletal support wreath;
generating a solid model of the fully individualized skeletal support wreath;
designing and positioning the slot of the fully individualized skeletal support wreath for the connecting girder in the frontal area of the wreath, wherein the center of the slot is located on the midsagittal plane;
designing and positioning reinforcement webs in the frontal and lateral areas of the fully individualized skeletal support wreath;
designing and positioning elliptical slits in the dorsal area of the fully individualized skeletal support wreath, determined at least through angles L and R when using axilla support and determined at least through angles L′ and R′ when using groin support, said elliptical slits adapted to fixate a pair of elastic straps;
generating a 3D solid model file of the digitally designed fully individualized skeletal support wreath;
placing the 3D solid model file of the fully individualized skeletal support wreath into a 3D printing slicer software; and
manufacturing the fully individualized skeletal support wreath.

41. A computer program comprising instructions which, when the program is executed by a computer, cause the computer to carry out a method, the method comprising:
instructions for defining a midsagittal plane including the Glabella, the Labrale superior and the Inion and a Frankfort horizontal plane including at least one of the following pairs of points: the left Orbitale with the left Tragion or the right Orbitale with the right Tragion, and being normal to the midsagittal plane, on a user;
instructions for generating a 3D reconstruction editable surface of at least part of the fully individualized skeletal support wreath's area on the head of the user;
instructions for creating the caudal and cranial boundaries of the fully individualized skeletal support wreath surface using as references the midsagittal and Frankfort horizontal planes and at least the points, lines and linear segments according to parameters 33-60, wherein parameters 33 to 60 are defined as:
parameter 33 is the craniometric point Glabella;
parameter 34 is the craniometric point Tragion;
parameter 35 is the craniometric point Orbitale;
parameter 36 is the Frankfort horizontal plane;
parameter 37 is the concha of external ear.
parameter 38 is the middle of the concha on the Frankfort horizontal plane;
parameter 39 is the craniometric point on the frontal bone contour located five to twenty mm above Glabella;
parameter 40 is the line crossing the craniometric point (parameter 39) and parallel to parameter 36;
parameter 41 is the perpendicular to the Frankfort horizontal plane through the craniometric point (parameter 34);
parameter 42 is the point where line of parameter 41 intersects line of parameter 40 and is the first control point of the spline curve of parameter 45;
parameter 43 is the perpendicular to the Frankfort horizontal plane through the point of parameter 38;
parameter 44 is the second control point of the spline curve of parameter 45, located at least one mm more caudally to the point of parameter 42 and on the line of parameter 43;
parameter 45 is the spline curve consisting of at least four control points and one free point at its end;
parameter 46 is the craniometric point Sub-inion right;
parameter 47 is the perpendicular to Frankfort horizontal plane crossing the craniometric point of parameter 46;
parameter 48 is the third control point of the spline curve of parameter 45, which is located on the perpendicular of parameter 47 and at least one mm above the craniometric point of parameter 46;
parameter 49 is the craniometric point Inion;
parameter 50 is the fourth control point of the spline curve of parameter 45, which is located at least one mm above the craniometric point of parameter 49 on the dorsal contour of the head;
parameter 51 is the free point of the spline curve of parameter 45 at its end;
parameter 52 is the craniometric point located on the frontal bone contour at least twenty mm above the craniometric point of parameter 39;
parameter 53 is the apex of the user's head;
parameter 54 is the perpendicular to Frankfort horizontal plane crossing the craniometric point of parameter 53;
parameter 55 is the point on the line of parameter 54, which is located on the craniometric point of parameter 53 or at least one mm below the craniometric point of parameter 53;
parameter 56 is the line parallel to Frankfort horizontal plane crossing the point of parameter 55;
parameter 57 is the craniometric point, which is located on the craniometric point of parameter 53 or on the dorsal contour of the user's head, where the line of parameter 56 intersects the head contour;
parameter 58 is the apex of the curve of the user's head between the craniometric points of parameters 49 and 57;
parameter 59 is the craniometric point, which is located on the craniometric point of parameter 53 or on the dorsal contour of the head located at least one mm above the craniometric point of parameter 58;
parameter 60 is a linear segment between parameters 52-59 representing the digital cranial boundaries of the fully individualized skeletal support wreath;
instructions for offsetting in an upward scaling to accommodate the hair volume of the user and the thickness of the interior soft inlay of the fully individualized skeletal support wreath;
instructions for generating a solid model of the fully individualized skeletal support wreath;
instructions for designing and positioning a slot of the fully individualized skeletal support wreath for the connecting girder in the frontal area of the wreath, wherein a center of the slot is located on the midsagittal plane;
instructions for designing and positioning reinforcement webs in frontal and lateral areas of the fully individualized skeletal support wreath;
instructions for designing and positioning elliptical slits in the dorsal area of the fully individualized skeletal support wreath, determined at least through angles L and R when using axilla support and determined at least through angles L′ and R′ when using groin support, said elliptical slits adapted to fixate a pair of elastic straps; and
instructions for generating a 3D solid model file of the digitally designed fully individualized skeletal support wreath.

42. The computer program of claim 41, wherein a 3D digital imprint of a head is obtained by use of a hybrid LED and Infrared Light Source Handheld Color 3D scanner.

43. The computer program of claim 41, wherein the fully individualized skeletal support wreath is formed as part of a protective helmet.

44. The computer program of claim 41, wherein the fully individualized skeletal support wreath is formed as part of a helmet for guiding symmetrical skull growth.
